(19) United States
(12) Patent Application Publication
(10) Pub. No.: US 2025/0197457 A1
(43) Pub. Date: Jun. 19, 2025

(54) CHIMERIC IGG-FC-BINDING LIGAND POLYPEPTIDE AND USES THEREOF FOR IGG AFFINITY PURIFICATION

(71) Applicant: Roche Diagnostics Operations, Inc., Indianapolis, IN (US)

(72) Inventors: Romina Eisenhauer, Penzberg (DE); Frank Kroner, Penzberg (DE); Jigar Patel, Pleasanton, CA (US); Michael Schraeml, Penzberg (DE); Martin Strauss, Penzberg (DE); Simone Taeuber, Penzberg (DE)

(21) Appl. No.: 18/433,619

(22) Filed: Feb. 6, 2024

Related U.S. Application Data
(63) Continuation of application No. PCT/EP2022/072045, filed on Aug. 5, 2022.
(60) Provisional application No. 63/230,305, filed on Aug. 6, 2021.

(30) Foreign Application Priority Data
Aug. 31, 2021 (EP) 21194146.3

(51) Int. Cl. C07K 14/195; C07K 1/22; C12N 1/20

(52) U.S. Cl. CPC C07K 14/195; C07K 1/22; C12N 1/20; C07K 2319/21

(57) ABSTRACT

The present invention relates to a chimeric IgG-Fc-binding ligand polypeptide, comprising a protein fragment of SlyD, wherein the IF domain thereof is replaced by the affinity peptide Fc-III-4C or an Fc-III-XC variant thereof, as well as related uses for IgG affinity purification.

### CROSS-REFERENCE TO RELATED APPLICATIONS

This application is a continuation of International PCT Application No. PCT/EP2022/072045 filed on Aug. 5, 2022, which claims priority to U.S. Provisional Patent Application No. 63/230,305 filed on Aug. 6, 2021 and European Patent Application No. 21194146.3 filed on Aug. 31, 2021, the contents of each application are incorporated herein by reference in their entireties.

### SEQUENCE LISTING

This application incorporates by reference the material in the ST.26 XML file titled Revised-2-Sequence-Listing-ROCHE-110, which was created on Dec. 19, 2024 and is 27,043 bytes.

The present invention relates to a chimeric IgG-Fc-binding ligand polypeptide, comprising a protein fragment of SlyD, wherein the IF domain thereof is replaced by the affinity peptide Fc-III-4C or an Fc-III-XC variant thereof, as well as related uses for IgG affinity purification.

### BACKGROUND OF THE INVENTION

Hand in hand with the success of monoclonal antibodies as therapeutic and diagnostic tools, the development of sufficient and economical IgG purification procedures became more necessary for both, academic and industrial use. Beside different types of interaction and separation techniques, affinity strategies have been the most common and efficient methodologies so far.

The growing implementation of antibodies has necessitated the development of suitable mAb purification strategies, especially for industrial-scale production. Compared to other biotechnological products several hundred kilograms of bulk drug substance of monoclonal antibodies have to be synthesized annually, due to the partially high dose administration and the broad indication field.

Streptococcal Protein A, a natural IgG-Fc binder, is still the gold standard among affinity-ligands at manufacturing scale. Coupled to an appropriate matrix, such as agarose beads, these affinity resins represent the first step in the purification chain to isolate antibodies from crude protein mixtures, such as serum, ascites fluid or cell supernatants, and therefore render further downstream-processes more economically.

The most commonly used process for downstream purification of monoclonal antibodies nowadays comprises a first step, where a producing cell culture is harvested via e.g. filtration in order to remove cells and cell debris and to yield a clarified supernatant suitable for chromatography. Afterwards the mAbs, present in the cell culture supernatant, are recovered in a single capture step by applying the filtered fluid to Protein A chromatographic columns. Process and product related impurities are removed by one or two polishing steps, typically incorporating cation or anion exchange chromatography, hydrophobic interaction chromatography or mixed mode chromatography.

Furthermore, several steps to bypass viral contamination such as viral inactivation after Protein A chromatography (e.g. a low pH incubation) and a viral filtration step are included in the process. The purified product is then transferred into the final formulation buffer via ultra- or diafiltration.

WO 2004/076485A1 describes a method of purifying an antibody by means of protein A affinity chromatography.

Although Protein A chromatography is highly effective in removing process related impurities and presents other remarkable features, such as high production yield and ease of operation, there are some drawbacks with regard to the general demands mentioned above: First, Protein A as a product of bacterial origin, can contaminate the final product by leaking from the column matrix and act as immunotoxin. Second, the costs are relatively high: On the one hand due to the resin production, since Protein A is obtained in a recombinant form from E. coli, and on the other hand for column regeneration: Protein A is not resistant to common cleaning and sanitizing agents, such as guanidine hydrochloride or urea, representing not only a cost- but also a disposal-challenge. Another serious cause of concern is the aggregate formation provoked by the antibody elution conditions at low pH values, which can lead to the loss of its biological activity (Arora, I., Chromatographic Methods for the Purification of Monoclonal Antibodies and their Alternatives: A Review. International Journal of Emerging Technology and Advanced Engineering, 2013. 3(10): p. 475-481).

One of the major drawbacks in conventional IgG purification processes is the elution at acidic pH values (up to pH 2.5-3.5). The high affinity of natural IgG binding proteins, such as protein A, towards their target results on the one hand in a high specificity and selectivity of the purification process, but on the other hand requires harsh conditions to disrupt the strong interaction between the antibody and the ligand. The low pH can promote the degradation of the mAbs and may lead to contamination of the final product due to column ligand leakage

To overcome these drawbacks of Protein A-based purification, a great effort was made in the improvement of alternative Ab-binding molecules, especially in view of binding properties as well as pH-sensitivity. Besides Protein A, which is mainly used for Ab isolation because of its high affinity towards IgGs from different species, other proteins of bacterial origin, such as Protein G, Z and L, have been investigated (Choe, W., T. A. Durgannavar, and S. J. Chung, Fc-Binding Ligands of Immunoglobulin G: An Overview of High Affinity Proteins and Peptides. Materials (Basel), 2016. 9(12)).

Affinity chromatographic techniques using Protein A and G resins are still the most widely used methods for mAb capture at manufacturing scale, since they represent a very well proven technology and are included in many established platform processes.

SlyD (sensitive to lysis D; product of the slyD gene) is a metallochaperon and consists of two domains representing two functional units: A peptidyl-prolyl cis/trans isomerase (PPIase) activity in the FK506 binding protein (FKBP) domain and a chaperon function in the 45-amino-acid ‘insert-in-flap’ (IF) domain (Low, C., et al., Crystal structure determination and functional characterization of the metallochaperone SlyD from Thermus thermophilus. J Mol Biol, 2010. 398(3): p. 375-90). Its superior biophysical characteristics, such as excellent thermal and proteolytic stability, caused by its origin from the thermophile organism Thermus thermophilus, makes the TtSlyD a powerful tool for a wide range of applications. A great advantage of the TtSlyD scaffold is that the IF domain can be easily exchanged without affecting the physiochemical behavior and tertiary structure of the FKBP core region. Moreover its simple structure and its high solubility, allow the recombinant production in bacterial cells, such as E. coli. However, therapeutic applications with the TtSlyD scaffold are not feasible in its original composition, due to its bacterial origin, triggering an immunological response.

WO 2003/000878A2 relates to the cloning and expression of a heterologous protein or polypeptide in bacteria such as Escherichia coli. In particular, this invention relates to expression tools comprising a FKBP-type peptidyl prolyl isomerase selected from the group consisting of FkpA, SlyD, and trigger factor, methods of recombinant protein expression, the recombinant polypeptides thus obtained as well as to the use of such polypeptides.

WO 2014/071978A1 is directed to a chimeric polypeptide protein scaffold for engineering polypeptide domains displayed by the scaffold comprising one or more fragments from the FKBP family displaying one or more polypeptides inserted in place of the insert-in-flap-domain (IF-domain) and its use in methods for the screening and selection of constrained peptide surrogates exhibiting binding activity versus predetermined target molecules, specifically a polypeptide of the sequence of MKVGQDKVVTIRYTLQVEGEVLDQGELSYLHGHRLIPGLEEALEGREEGEAFQAH VPAEKAY-X-GKDLDFQVEVVKVREATPEELLHGHA (SEQ ID NO:2), wherein X is an amino acid sequence comprising a variable sequence to be displayed by the Thermus thermophilus SlyD chimeric polypeptide.

It is an object of the present invention to provide an improved IgG purification procedure, including a capture system that exhibits a fast complex formation between the ligand and the target analyte and allows dissociation under relatively mild conditions, and wherein the method also avoids further disadvantages of the prior art. Other objects and advantages will become apparent to the person of skill when studying the present description of the present invention.

In a first aspect of the present invention, the above object is solved by a chimeric IgG-Fc-binding ligand polypeptide, comprising a protein fragment of SlyD, wherein the IF domain thereof is replaced by the affinity peptide Fc-III-4C (CDCAWHLGELVWCTC, SEQ ID NO: 1) or a Fc-III-XC variant thereof (X₁DCAWHLGELVWCTX₂, SEQ ID NO: 3), wherein X₁ is either missing or independently selected from the group of C, D, P, E, and K, and X₂ is independently selected from the group of C, Q, P, and E. Preferably, the chimeric IgG-Fc-binding ligand polypeptide according to the present invention comprises SlyD from a Thermus species, such as, for example, Thermus thermophiles.

In a second aspect of the present invention, the above object is solved by a divalent binder molecule, comprising two fused chimeric IgG-Fc-binding ligand polypeptides according to the present invention that are preferably fused head-to-tail with each other.

In a third aspect of the present invention, the above object is solved by the chimeric IgG-Fc-binding ligand polypeptide according to the present invention or the divalent binder molecule according to the present invention that is coupled to a solid carrier, such as a solid matrix material, such as a bead and/or column matrix.

In a fourth aspect of the present invention, the above object is solved by a method for producing the chimeric IgG-Fc-binding ligand polypeptide according to the present invention, comprising recombinant expression of said ligand polypeptide in a suitable host cell, such as E. coli, or comprising a chemical synthesis of said ligand polypeptide.

In a fifth aspect of the present invention, the above object is solved by a method for purifying an immunoglobulin, comprising contacting a solid carrier having the chimeric IgG-Fc-binding ligand polypeptide according to the present invention or the divalent binder molecule according to the present invention coupled thereto with said immunoglobulin, and suitably eluting said immunoglobulin from said chimeric IgG-Fc-binding ligand polypeptide or the divalent binder molecule, wherein said method preferably comprises fast protein liquid chromatography (FPLC).

In a sixth aspect of the present invention, the above object is solved by the use of the chimeric IgG-Fc-binding ligand polypeptide according to the present invention or the divalent binder molecule according to the present invention for purifying immunoglobulins, or for screening and selecting of peptide binders against predetermined target molecules.

In a main aspect of the present invention a new type of binder and respective immuno-affinity chromatographic column material, applicable for IgG purification, was developed. In a particularly preferred aspect, the new material is suitable for immunoglobulin affinity purification via fast protein liquid chromatography (FPLC). The present invention provides an effective, less cumbersome and cost-efficient alternative to Protein A.

The present invention provides a chimeric IgG-Fc-binding ligand polypeptide, comprising a protein fragment of SlyD, wherein the IF domain thereof is replaced by the affinity peptide Fc-III-4C (CDCAWHLGELVWCTC, SEQ ID NO: 1).

The comprised Fc-binding ligand was based on the existing IgG-Fc affinity peptide, called Fc-III-4C, described by Gong et al. in 2016 (Gong, Y., et al., Development of the Double Cyclic Peptide Ligand for Antibody Purification and Protein Detection. Bioconjug Chem, 2016. 27(7): p. 1569-73). By grafting the peptide onto the FKBP domain of the Thermus thermophilus chaperon SlyD, surprisingly, an extremely stable and easy-to-produce affinity ligand called TtSlyD-Fc-III-4C was obtained. Furthermore, chromatographic columns were generated by covalently immobilizing this TtSlyD-Fc-III-4C chimera on NHS-activated sepharose resins, and successfully tested.

The crystallographic structure of the domain B of SpA binding to the IgG-Fc-portion was resolved in 1981 and revealed the interaction site with the CH2 and the CH3 domain of the immunoglobulin. The subunit B contains two alpha-helices, and eleven residues participate in the binding process. The respective binding site on the Fc-fragment is also the point of contact for other natural binding proteins, such as Protein G and the neonatal Fc-receptor and appears to be a preferred point for protein-protein interactions due to its physiochemical properties. This specific interaction site was targeted by DeLano and coworkers in 2000 (DeLano, WI., et al., Convergent solutions to binding at a protein-protein interface. Science, 2000. 287(5456): P. 1279-83). By screening a cyclic peptide library via phage-display, they identified a 13-residue sequence (DCAWHLGELVWCT, SEQ ID NO: 4) binding to the consensus site of the IgG-Fc-region which was found to compete with natural binders. X-ray crystal structure analysis then revealed an alpha-hairpin conformation of the selected Fc-III peptide. Despite its completely different structure to known binding motifs and its four-times smaller size, Fc-III and Protein A share many intermolecular interaction points on the surface of the Fc-part and the binding affinity of the Fc-III peptide was only about two fold weaker.

In 2006, Dias and coworkers tried to further stabilize the hairpin conformation by restricting the conformational freedom of the peptide (Dias, R. L., et al., Protein ligand design: from phage display to synthetic protein epitope mimetics in human antibody Fc-binding peptidomimetics. J Am Chem Soc 2006. 128(8): p. 2726-32). They created two backbone cyclic peptidomimetics based on the original Fc-III. The resulting FcBP-1 and FcBP-2 peptides were generated by grafting Ala3-Trp11 or Asp1-Thr13 onto a hairpin-inducing D-Pro-L-Pro template. FcBP-2 showed an 80-fold higher affinity for the Fc-domain compared to Fc-III. This was attributed to backbone cyclisation and the constraintment by the additional disulfide bridge. FcBP-1 in contrast, which lacks the crucial disulfide bridge, interacted only weakly with the Fc-domain. In 2012, the attempt of stabilizing the peptide in a double cyclic structure was further processed by Gong and coworkers (Gong, Y., et al., Development of the Double Cyclic Peptide Ligand for Antibody Purification and Protein Detection. Bioconjug Chem, 2016. 27(7): p. 1569-73).

Furthermore, to simplify the expression and purification of the TtSlyD-FcIII-4C scaffolds, derivatives of the molecule were designed, and chemically synthesized peptides were tested for binding against IgG (mAbs) in a high-throughput microarray. Both cysteine residues, forming the disulfide bridges at the stalk of the emerging loop structure, were either exchanged or completely removed.

Because of difficulties in synthesizing the D-Pro-L-Pro backbone, the two prolines were replaced by two Cysteine residues at the N- and C-termini intending to form a second disulfide linkage. The newly generated peptide, termed Fc-III-4C (CDCAWHLGELVWCTC, SEQ ID NO: 1), was analyzed via surface plasmon resonance (SPR) and showed a 30-fold higher binding affinity to human IgG compared to the original Fc-III. Furthermore, it showed strong interactions to a variety of IgGs from different species. Its potential as ligand for antibody purification was confirmed by selectively capturing IgG from rabbit sera with agarose beads carrying the immobilized affinity peptide.

The Fc-III-4C peptide beads showed an even higher binding capacity and reusability than commercial protein A beads. The crystal structure of the Fc-III peptide in complex with the IgG-Fc-domain shows that the constrained peptide loop targets the same binding site as natural Fc-binders. It interacts with similar amino acids that were found in the interface of commonly used Fc-binding proteins.

Furthermore, the complex stability of the Fc-III-4C binding peptide-IgG-Fc complex was altered through systematically engineering the Fc-III-4C loop insertion. One of the amino acids that seems to play a key role in the complex formation is Trp11, and the respective residue was exchanged against twelve other amino acids, exhibiting different chemical properties. The resulting mutants were expressed, purified and tested via SPR analysis. The findings as obtained were adopted and tested with the present TtSlyD scaffold platform. They resulted in chimeric IgG-Fc-binding ligand polypeptides according to the present invention, comprising a protein fragment of SlyD, wherein the IF domain thereof is replaced by the affinity peptide Fc-III-XC thereof (X₁DCAWHLGELVWCTX₂, SEQ ID NO: 3), wherein X₁ is either missing or independently selected from the group of C, D, P, E, and K, and X₂ is independently selected from the group of C, Q, P, and E.

Peptide-based ligands became a promising new class of binders and have been successfully developed by different research groups. With the prospect of using them alternatively to Protein A, they can be produced at lower costs and are, compared to natural binders, non-immunogenic. Within this group of binders, especially cyclic peptides have some promising features for the use as affinity ligands. Compared to their linear counterparts, they exhibit a higher enzymatic stability and conformational rigidity, leading to a higher specificity and/or affinity towards their target and an entropic advantage in binding. Nevertheless, a chemical synthesis of the peptide is still necessary, and aggregation during synthesis may lead to low yields.

For the chimeric IgG-Fc-binding ligand polypeptide according to the present invention, any suitable SlyD scaffold protein can be used, and is preferably from a bacterial species, such as SlyD from E. coli, as well as SlyD orthologues from Yersinia pestis, Treponema pallidum, Pasteurella multocida, and Vibrio cholerae. Further preferred is SlyD from a Thermus species, such as, for example, Thermus thermophiles.

More preferred is the chimeric IgG-Fc-binding ligand polypeptide according to the present invention, comprising the sequence

(SEQ ID NO: 5)
MKVGQDKVVTIRYTLQVEGEVLDQGELSYLHGHRLIPGLEEALEGREEG
EAFQAHVPAEKAYCDCAWHLGELVWCTCGKDLDFQVEVVKVREATPEEL
LHGHA.

Further preferred is the chimeric IgG-Fc-binding ligand polypeptide according to the present invention, wherein the IF domain thereof is replaced by the affinity peptide Fc-III-XC selected from the group consisting of
DCAWHLGELVWCTX₂, SEQ ID NO: 6
CDCAWHLGELVWCTX₂, SEQ ID NO: 7
DDCAWHLGELVWCTX₂, SEQ ID NO: 8
PDCAWHLGELVWCTX₂, SEQ ID NO: 9
EDCAWHLGELVWCTX₂, SEQ ID NO: 10, and
KDCAWHLGELVWCTX₂, SEQ ID NO: 11, wherein X₂ is independently selected from the group of C, Q, P, and E, and/or
selected from the group consisting of

SEQ ID NO: 12
X₁DCAWHLGELVWCTC,
SEQ ID NO: 13
X₁DCAWHLGELVWCTQ,
SEQ ID NO: 14
X₁DCAWHLGELVWCTP,,
and
SEQ ID NO: 15
X₁DCAWHLGELVWCTE,,

wherein X₁ is either missing or independently selected from the group of C, D, P, E, and K.

Even further preferred is the chimeric IgG-Fc-binding ligand polypeptide according to the present invention, wherein the IF domain thereof is replaced by the affinity peptide Fc-III-XC having the sequence EDCAWHLGELVWCTE, SEQ ID NO: 16 (TtSlyD-Fc-III-2C Hit No. 4).

Still further preferred is the chimeric IgG-Fc-binding ligand polypeptide according to the present invention, further comprising a C-terminal amino acid tag, such as, for example, a Hiss tag. The proteins harbouring an affinity-tag consisting of polyhistidine residues are advantageously captured due to the interaction between the metal-ions (Ni²⁺) immobilised on the matrix and the 8×-histidine side chains (see examples).

The chimeric IgG-Fc-binding ligand polypeptide according to the present invention further has the advantage over protein A/G that the polypeptide exhibits binding with high affinity to broad IgG species, e.g. selected from the group consisting of human, rabbit (see examples), mouse, rat, pig, goat, horse, and bovine IgG.

Protein ligand multimerisation can lead to an even higher alkaline stability and improved binding capacities of the affinity column matrices due to avidity effects. Therefore, a divalent binder, with a molecular weight of 29 kDa was generated, by head-to-tail linkage of the TtSlyD-Fc-III-4C with a second scaffold protein, exhibiting the same Fc-III-4C peptide loop. This dual binder showed an even higher affinity towards the Fc-part of IgG, compared to the single scaffold, in an SPR interaction assay (KD=5 nM, for rbIgG at 37° C. and human IgG at 25° C.). Therefore, another preferred aspect of the present invention relates to a divalent binder molecule, comprising two fused chimeric IgG-Fc-binding ligand polypeptides according to the present invention, that preferably are fused head-to-tail with each other.

Yet another aspect of the present invention relates to the chimeric IgG-Fc-binding ligand polypeptide according to the present invention, or the divalent binder molecule according to the present invention, coupled to a solid carrier, such as a solid matrix material, such as a bead, such as an agarose bead and/or a column matrix, preferably an NHS-activated matrix, such as sepharose resins. Preferred is the coupled ligand or binder molecule according to the present invention, wherein said coupling is through the lysine side chains of the SlyD with NHS esters comprised in a sepharose matrix.

Yet another aspect of the present invention relates to the solid carrier material as above, such as a bead, such as an agarose bead and/or a column matrix, preferably an NHS-activated matrix, such as sepharose resins, coupled with the chimeric IgG-Fc-binding ligand polypeptide according to the present invention, or the divalent binder molecule according to the present invention. The material has improved properties, such as less leakage and increased reusability as described herein. Yet another aspect of the present invention relates to an immuno-affinity chromatographic column comprising the solid carrier material according to the present invention, which preferably is applicable for IgG purification also as disclosed herein.

Yet another aspect of the present invention relates to a method for producing the chimeric IgG-Fc-binding ligand polypeptide according to the present invention, comprising recombinant expression of said ligand polypeptide in a suitable host cell, such as E. coli, or comprising a chemical synthesis of said ligand polypeptide. Respective methods are known in the art and disclosed herein as well. Preferred is the method according to the present invention, further comprising the step of coupling the chimeric IgG-Fc-binding ligand polypeptide to a solid carrier, such as a solid matrix material, such as a bead and/or column matrix, in particular to a matrix as mentioned above. The matrix can be packed into a suitable column.

Still another aspect of the present invention relates to a method for isolating or purifying an immunoglobulin, comprising contacting a solid carrier having the chimeric IgG-Fc-binding ligand polypeptide according to the present invention or the divalent binder molecule according to the present invention coupled thereto with said immunoglobulin, and suitably eluting said immunoglobulin from said chimeric IgG-Fc-binding ligand polypeptide or the divalent binder molecule. Preferred is the method according to the present invention, comprising fast protein liquid chromatography (FPLC). Exemplary methods are disclosed in the present examples, and in the literature for affinity chromatography, such as Elliott L. Rodriguez, et al. (Affinity chromatography: A review of trends and developments over the past 50 years, Journal of Chromatography B, Volume 1157, 2020, https://doi.org/10.1016/j.jchromb.2020.122332 or Huse, Klaus & Bohme, Hans-Joachim & Scholz, Gerhard. (2002). Purification of antibodies by affinity chromatography. Journal of biochemical and biophysical methods. 51. 217-31. 10.1016/S0165-022X(02)00017-9). The methods can be performed re-using the matrix materials as disclosed herein, and can be adjusted to industrial scale production.

Preferred is the method according to the present invention, wherein the elution conditions for said immunoglobulin are milder compared to a solid carrier material comprising protein A coupled thereto. In particular in case of the variants, a fast association rate, favorable in terms of antibody capture, and a fast dissociation rate allow for antibody detachment under relatively mild pH conditions. With protein A, low pH, e.g. acetic acid with a pH-value of about 3.3 has been used as a standard procedure for elution (Gûlich, S., M. Uhlen, and S. Hober, Protein engineering of an IgG-binding domain allows milder elution conditions during affinity chromatography. J Biotechnol, 2000. 76(2-3): p. 233-44). The low pH can promote the degradation of the mAbs and may lead to contamination of the final product due to column ligand leakage. A preferred pH elution range for the present invention is between 3.4 and 4.5.

Also preferred is the method according to the present invention, comprising a chemical regeneration step for said solid carrier material using harsher conditions compared to a solid carrier material comprising protein A coupled thereto. A successful regeneration involves the removal of the bound analyte without detaching the ligand or restricting its activity. The protein A/G binding to antibodies is relatively strong, and regeneration requires washing with, e.g., glycine buffer, pH 2.7. A preferred pH regeneration range for the present invention is between 2.5 and 2.0.

Yet another aspect of the present invention relates to the use the chimeric IgG-Fc-binding ligand polypeptide according to the present invention or the divalent binder molecule according to the present invention for purifying immunoglobulins, or for screening and selecting of peptide binders against predetermined target molecules.

The chemically synthesised, so called Fc-III-4C peptide is constrained by two disulfide bridges to favor a binding-competent-hairpin conformation that precisely complements its target. Investigations were based on a phage-display-identified cyclic 13-mer peptide, that was first described by De-Lano and coworkers in 2000 (DeLano, W. L., et al., Convergent solutions to binding at a protein-protein interface. Science, 2000. 287(5456): p. 1279-83) and was further optimised in regards to conformational stability and binding affinity (KD=2.45 nM for human IgG) by Gong et al. in 2016 (Gong, Y., et al., Development of the Double Cyclic Peptide Ligand for Antibody Purification and Protein Detection. Bioconjug Chem, 2016. 27(7): p. 1569-73).

In comparison to the two disulfide bridges necessary in the constrained peptide synthesis, the scaffold binder needs only one bridge to be functional which facilitates the cheap recombinant production in E. coli with high yields. In comparison to Protein A, harsher chemical regeneration conditions are possible for affinity columns, and milder elution conditions of antibodies compared to protein A.

In the present invention, the feasibility and efficiency of a new type of immuno-affinity chromatographic columns, applicable for IgG purification, were demonstrated for the first time. It was shown, that grafting the Ig-Fc affinity peptide Fc-III-4C onto the TtSlyD scaffold protein is viable, while retaining the peptide's specificity and affinity.

Advantages of the single Fc-III-4C affinity peptide over protein A/G are that it exhibits a high affinity towards many IgG species (human, rabbit, mouse, rat, pig, goat, horse, bovine) and, as an affinity ligand for mAb purification, immobilised on agarose beads, Fc-III-4C shows, compared to standard protein A beads, an extended reusability. Furthermore, the TtSlyD-Fc-III-4C scaffold protein, coupled to NHS-sepharose, possesses an excellent chemical robustness—proven by repeatedly denaturing and refolding the protein—and is highly tolerant towards alkaline treatment.

Affinity chromatographic columns comprising TtSlyD-Fc-III-4C are an economical and efficient alternative to alkaline-sensitive Protein A matrices. In addition, the protein scaffolds provide a high conformational stability of the displayed peptide loop and an enhanced protease resistance, resulting in a low ligand-leakage and a low contamination of the final product. Moreover, the scaffold proteins can be generated quickly and cost-effectively in large amounts by recombinant expression in E. coli. Approximately 600 mg biomass and a final protein yield of >5 mg per 100 mL bacterial cell culture were obtained for all expressed protein variants.

Furthermore, incorrect folding and oligomerisation of the proteins, mainly driven by unspecific, intermolecular disulfide bridge formation, are a major issue. By eliminating two terminal cysteine residues that are constraining the peptide loop, it was shown that the synthesis could be improved. Variants exhibiting only a single disulfide linkage, TtSlyD-Fc-III-XC, were less prone to aggregation compared to the wild type scaffold, harboring four cysteines.

Furthermore, to overcome the issue of acidic elution conditions, the binding site of the TtSlyD-Fc-III ligand could be further engineered. The hot spot residue leucine 7, may be modified in order to obtain the desired association/dissociation properties.

In the context of the present invention, the Fc-binding part of the peptide was furthermore altered by QC-PCR as described below, intending to selectively screen for protein variants that exhibit the following features: A fast association rate, favorable in terms of antibody capture and a fast dissociation rate, allowing for antibody detachment under relatively mild pH conditions. Surface plasmon resonance was used in order to determine the binding affinities of the generated TtSlyD-Fc-III-4C variants towards IgG. For doing so, scaffolds were immobilized on a Biacore chip, interacting with IgG in a flow cell as described below. The analyte (IgG) was applied in five different concentrations and each concentration was monitored in a single cycle, regenerating the chip surface after each run. A regeneration scouting with human IgG was performed prior to the kinetic measurements, in order to check the best conditions for completely removing the analyte after each cycle. With respect to the results obtained in this experiment, it can be concluded that grafting the Fc-III-4C peptide onto the TtSlyD domain does not negatively affect its affinity to IgG-Fc. Moreover, no protein variants exhibiting altered kinetic properties, such as faster association or dissociation rates, could be detected, by exchanging the tryptophan residue at position 11. A possible explanation for that might be, that the tryptophan's contribution to the binding energy is lower than initially expected. In future investigations the focus could be shifted to another amino acid, showing a more significant contribution to the binding energy. For example, the leucine at position 7 in the Fc-III-4C peptide, represents a typical hot spot at protein-peptide interfaces. Furthermore, in the following experiments it has been shown, that by exchanging the respective residue the binding strength can be influenced considerably.

The four cysteine residues, present in the Fc-III-4C peptide loop, form the cyclic structure and define the rigidity and affinity of the binder. However, cysteines represent a major issue in terms of protein expression and purification. Dias et al. have already demonstrated that by removing the disulfide linkage in the Fc-III peptide, a complete loss of binding affinity occurs (Dias, R. L., et al., Protein ligand design: from phage display to synthetic protein epitope mimetics in human antibody Fc-binding peptidomimetics. J Am Chem Soc, 2006. 128(8): p. 2726-32). In the present invention, structural stability of the affinity peptide is preserved by the TtSlyD backbone, and the extra disulfide link may not be needed. In the following experiments, the cysteines were replaced by alternative amino acids, in order to investigate whether these linkages are essential for the loop stability and protein's affinity towards the IgG-Fc.

In the context of the present invention, the feasibility and efficiency of a new type of immuno-affinity chromatographic columns, applicable for IgG purification, were demonstrated for the first time. It was shown, that grafting the Ig-Fc affinity peptide Fc-III-4C onto the TtSlyD scaffold protein is viable, while at least retaining the peptide's specificity and affinity.

Advantages of the single Fc-III-4C affinity peptide over protein A/G are that it exhibits a high affinity towards many IgG species (human, rabbit, mouse, rat, pig, goat, horse, bovine) and, as an affinity ligand for mAb purification, immobilised on agarose beads, Fc-III-4C shows, compared to standard protein A beads, an extended reusability. Furthermore, the TtSlyD-Fc-III-4C scaffold protein, coupled to NHS-sepharose, possesses an excellent chemical robustness—proven by repeatedly denaturing and refolding the protein—and is highly tolerant towards alkaline treatment. Affinity chromatographic columns comprising TtSlyD-Fc-III-4C thus is an economical and efficient alternative to alkaline-sensitive Protein A matrices. In addition, the protein scaffolds provide a high conformational stability of the displayed peptide loop and an enhanced protease resistance, resulting in a low ligand-leakage and a low contamination of the final product.

Moreover, the scaffold proteins can be generated quickly and cost-effectively in large amounts by recombinant expression in E. coli. Approximately 600 mg biomass and a final protein yield of about 5 mg per 100 mL bacterial cell culture were obtained for all expressed protein variants. The generated amounts were sufficient for the screening experiments performed in this thesis, but could be further optimised in terms of yield and solubility for a potential large-scale production, e.g. by lowering the cultivation temperature or the amount of inducer substance (Marisch, K., et al., Evaluation of three industrial Escherichia coli strains in fed-batch cultivations during high-level SOD protein production. Microb Cell Fact, 2013. 12: p. 58). Furthermore, incorrect folding and oligomerisation of the proteins, mainly driven by unspecific, intermolecular disulfide bridge formation, are a major issue. By eliminating two terminal cysteine residues that are constraining the peptide loop, it was shown that the synthesis could be improved. Variants exhibiting only a single disulfide linkage, TtSlyD-Fc-III-XC, were less prone to aggregation compared to the wild type scaffold, harboring four cysteines.

Protein ligand multimerisation can lead to an even higher alkaline stability and improved binding capacities of the affinity column matrices due to avidity effects. Therefore, a divalent binder, with a molecular weight of 29 kDa was generated, by head-to-tail linkage of the TtSlyD-Fc-III-4C with a second scaffold protein, exhibiting the same Fc-III-4C peptide loop. This dual binder showed an even higher affinity towards the Fc-part of IgG, compared to the single scaffold, in an SPR interaction assay (KD=5 nM, for rbIgG at 37° C. and human IgG at 25° C.). However, production and purification of this dual binder is more complicated, in regards of protein aggregation and conjugation.

Furthermore, to overcome the issue of acidic elution conditions, the binding site of the TtSlyD-Fc-III ligand could be further engineered. The hot spot residue leucine 7 could be further modified in order to obtain the desired association/dissociation properties.

The present invention will now be described further in the following examples with reference to the accompanying Figures, nevertheless, without being limited thereto. For the purposes of the present invention, all references as cited herein are incorporated by reference in their entireties. In the Figures,

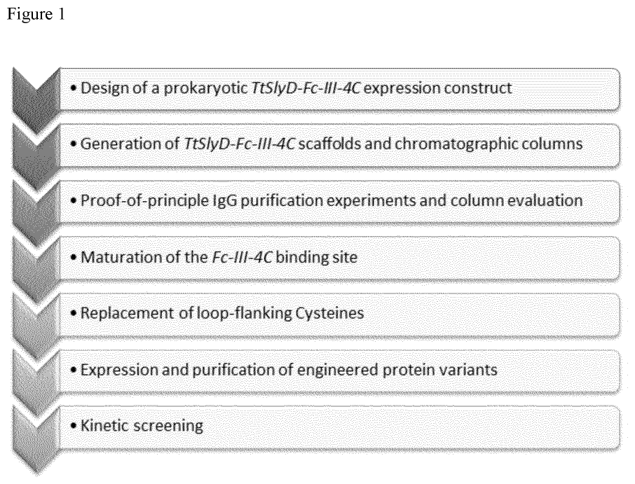

FIG. 1 shows the general workflow in the present invention.

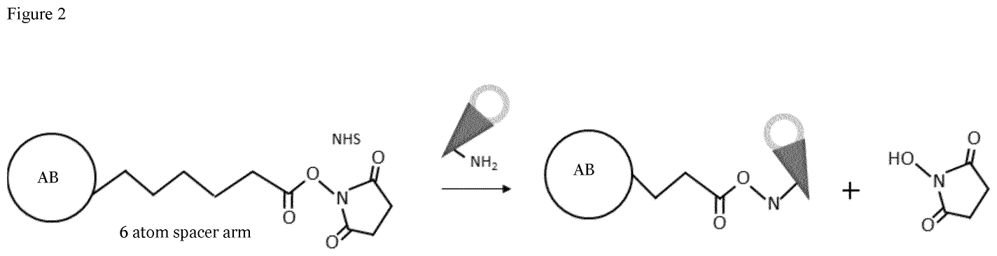

FIG. 2 shows an example for the coupling process of the inventive binders to a matrix. The column as used is comprised of NHS esters attached to sepharose HP via six-atom spacer arms. The activated esters react rapidly with ligands containing primary amino groups resulting in a very stable amide linkage. AB=agarose bead

### EXAMPLES

As a general outline, in the context of the present invention, a prokaryotic TtSlyD-Fc-III-4C expression construct was designed, and TtSlyD-Fc-III-4C scaffold molecules and chromatographic columns were generated. Then, IgG purification experiments were conducted. Subsequently, a maturation of the Fc-III-4C binding site was performed, loop-flanking cysteines were replaced, followed by a kinetic screening of engineered protein variants. Suitable variants were selected.

Molecular Cloning of E. coli Expression Constructs

The cloning of the TtSlyD-Fc-III-4C scaffold-affinity peptide chimers was performed in a two-step reaction. First, a gene coding for the TtSlyD protein backbone and an 8×-histidine-tag was inserted into a prokaryotic expression vector using the restriction endonucleases EcoRI-HF® and HindIII-HF®. Subsequently the sequences encoding the appropriate affinity peptides were integrated in the respective insertion site of the TtSlyD scaffold via BsiWI-HF® and BamHI-HF®. For the molecular grafting and generation of TtSlyD-Fc-III-XC expression constructs, double-stranded linear DNA fragments (so called DNA strings) were designed, containing the desired variations in the appropriate site of the TtSlyD sequence, and directly cloned into the prokaryotic expression vector pQE80-Kan.

The expression vectors used in this invention, pQE80-Kan-TtSlyD-Fc-III-4C and pQE80-Kan-TtSlyD-Fc-III-XC, are derived from the pQE80-Kan vector and contain the following features: KanR: Antibiotic resistance against Kanamycin; ColEI: Origin of replication; lacIq: lac repressor; PT5: T5 promotor (originating from coliphage T5); MCS: Multiple cloning site with restriction sites i.a. for EcoRI and HindIII.

The expression system used in this invention relies on the inducible T5-lac system [65]. In the absence of lactose, the lac repressor protein lacIQ encoded in the expression vector, blocks the bacterial RNA polymerase (RNAP) from binding to the promoter of the lac operon. Isopropyl-beta-D-thiogalactoside (IPTG), a structural non-metabolisable analogue of allolactose that binds and inactivates laqIQ, allows the RNAP to transcribe the sequences downstream of the T5 promotor (PT5). The generated transcripts can then be translated into recombinant protein.

### Quick Change PCR (QC-PCR)

The exchange of Trp11 was performed via Quick change polymerase chain reaction (QCPCR, described by Braman et al. (Braman, G.P.C.G.A., Site-directed mutagenesis using double-stranded plasmid DNA templates. Methods Mol Biol, 1996. 57: p. 31-44) on the expression construct pQE80-Kan-TtSlyDFc-III-4C according to the instructions of the QuikChange™ site directed mutagenesis kit (Agilent). Therefore, two mutagenic primers for each exchange were designed complementary to the targeted plasmid site. Both, forward and reverse primer, contained the desired mutation and anneal to the same position on the opposite strand of the plasmid. During QC-PCR, the mutagenic primers were extended using the non-strand-displacing Cloned Pfu DNA polymerase (Agilent), resulting in nicked circular strands. By adding the restriction enzyme DpnI (NEB) to the finalised PCR reaction mix, the non-mutated parental DNA template, which is recognised by its methylation sites, was digested. The nicked dsDNA was transformed into E. coli cells, where nicks are repaired during replication. Primers were designed according to the Agilent kit instructions mentioned above with 25-45 bases in length (37 bases for the forward and 41 bases for the reverse primers) and melting temperatures (Tm) of about 78° C. The desired mutation had to be in the middle of the primer with ˜10-15 bases of correct sequence on both sides. All primers were synthesised by Metabion, Planegg-Steinkirchen.

The QC-PCR reaction mixture was combined on ice in 0.2 mL reaction tubes. Cloned Pfu DNA polymerase was added shortly before starting the PCR amplification. Thermocycling conditions were adjusted to the length of the DNA template and the type of desired mutation. For a single amino acid exchange in the 4.8 kb plasmid an extension time of 10 min and 16 PCR cycles were chosen. A negative control without template DANN was prepared for each reaction. Following temperature cycling, 2 μL DpnI were added directly to each amplification reaction in order to digest the parental plasmid DNA. Samples were incubated for 1 h at 37° C. followed by a 20 min heat inactivation step of the restriction enzyme at 80° C. PCR samples were subsequently analysed on a 1% (w/v) agarose-gel.

Desired bands were excised, and the plasmid DNA was purified and transformed into NEB® Express competent E. coli (high efficiency) cells as described. One single colony of each construct was picked and DNA was isolated. Sequence analyses were carried out in order to verify inserted mutations.

Small-Scale Protein Test Expression Small-scale test expression experiments of the various TtSlyD-Fc-III-4C proteins were carried out to assess the amount of produced protein and solubility of the different variants. The expression (NEB® Express competent E. coli (high efficiency), BL21 derivate) was implemented in 96-well deep-well-blocks (DWB). Overnight starter cultures were prepared by inoculating 1.2 mL LB media (50 μg/mL Kanamycin) from glycerol stocks. Plates were incubated at 37° C. on an orbital shaker at 750 rpm for 16 h. The next day, expression cultures (1.2 mL of fresh Super Broth (SB) media) were inoculated with 50 μL of the pre-culture. Upon achieving the exponential growth phase (OD600 ˜1-1.2) expression was induced by adding IPTG to a final concentration of 0.5 mM. After cultivation for five hours at 37° C. the cells were harvested by centrifugation (4000 rcf, 20 min). Prior to inoculation and prior to cell harvest, samples for subsequent SDS-PAGE analysis were taken, respectively (Pre-Induction, PreI and Post-Induction, PostI). Optical densities of the bacterial cultures were determined and sample volumes were adjusted. The cell suspensions were centrifuged (8000 rcf, 3 min) and cells were resuspended in 32.5 μL 10 mM Tris pH 8.0, 4% SDS, 2% 2-Mercaptoethanol to obtain crude cell extracts, applicable to SDS-PAGE.
Mechanical Cell Disruption Cell pellets were resuspended in 800 μL Bead Ruption Buffer and transferred into a 2 mL tube filled with 150 mg 0.1-0.2 mm of glass beads. The solution was mixed with 50 μL of a 1:1000 dilution of Antifoam 204 (Sigma-Aldrich) and cells were disrupted mechanically using the Bead Ruptor 24 (Omni International) according to the following program. 100 μL of the bacterial lysate were transferred into a 2 mL tube and centrifuged (8000 rcf, 3 min), in order to separate the soluble from the insoluble fraction. The insoluble fraction was resuspended in 100 μL Bead Ruption Buffer prior to application to SDS-PAGE. Protein expression level and solubility were assessed via SDS-PAGE.

### Mid-Scale Protein Expression

In order to obtain a sufficient amount of biomass for subsequent protein purification, midscale expression cultures of the different TtSlyD scaffold variants were prepared. 5 mL overnight cultures (inoculated from glycerol stocks) of the respective mutants were used to inoculate 250 mL cultures of SB media containing 50 μg/mL Kanamycin. Cultures were incubated at 37° C. and 250 rpm in a shaking incubator until they reached an OD600 of 1-1.2. Expression was induced by adding IPTG to a final concentration of 0.5 mM. After cultivation for another five hours, cells were harvested by centrifugation (6000 rcf, 20 min). 2 mL-samples of the final cell suspension were taken and cells were disrupted mechanically to obtain samples for subsequent SDS-PAGE analysis.

### Chemical Cell Disruption

Cell pellets were lysed by adding 2 mL B-PER II™ Bacterial Protein Extraction Reagent (Thermo Fisher Scientific) per mg cells. Protease inhibitor phenylmethylsulfonyl fluoride (PMSF, 3 μL of 0.1 M per mL B-PER II™, Thermo Fisher Scientific) and DNaseI (Roche, one spatula tip per mL B-PER II™, Roche) were added, in order to prevent protein degradation and to reduce viscosity of the lysates, caused by unwanted DNA. The suspensions were incubated for 30 min on wet ice and filled up to a final volume of 20 mL with 20 mM Tris, 150 mM NaCl, pH 7.5 (Buffer A). The lysates were centrifuged for 15 min at 5000 rcf to separate the soluble from the insoluble fraction. Samples for SDS-PAGE were taken after resuspending the insoluble fraction in 20 mL Buffer A. The supernatants containing soluble proteins were further purified by Ni²⁺ affinity and size-exclusion chromatography.

### Protein Purification

### Immobilised Metal-Ion Affinity Chromatography (IMAC)

Scaffold proteins were initially purified via IMAC, first formulated by Porath et al. in 1975 (Porath, J., et al., Metal chelate affinity chromatography, a new approach to protein fractionation. Nature, 1975. 258(5536): p. 598-9). After cell lysis and centrifugation, the supernatants containing the soluble protein fraction were sterile-filtered (0.2 μm) and directly applied to nickel-charged nitrotriacetic acid (Ni-NTA) gravity flow columns (Pierce Gravity Flow Columns and Filter Units filled with Qiagen Superflow Ni-NTA agarose resin; column volume (CV) ˜1 mL). The proteins harbouring an affinity-tag consisting of polyhistidine residues are captured due to the interaction between the metal-ions (Ni²⁺) immobilised on the matrix and the 8×-histidine side chains. Buffers and solutions used for purification are as follows.

Buffers and solutions used for gravity flow purification. All buffers and solutions were sterile-filtered (0.2 μm) and degassed prior to use. The pH was adjusted either with HCl or NaOH.

### Buffer/Solution Composition

Buffer A (Sample Buffer) 20 mM Tris, 150 mM NaCl, pH 7.5
Buffer B1 (Washing Buffer) 20 mM Tris, 150 mM NaCl, 10 mM Imidazole, pH 7.5
Buffer B2 (Washing Buffer) 20 mM Tris, 150 mM NaCl, 50 mM Imidazole, pH 7.5
Buffer C (Elution Buffer) 20 mM Tris, 150 mM NaCl, 250 mM Imidazole, pH 7.5
Buffer D (Regeneration Buffer) 20 mM Tris, 150 mM NaCl, 500 mM Imidazole, pH 7.5
Stripping Solution 50 mM EDTA, 1% SDS, pH 7.5
Recharging Solution 100 mM NiSO₄
Storage Solution 20% Ethanol

Prior to use, columns were rinsed with filtered and degassed ddH₂O. Equilibration was performed with 20 CV of Buffer A, followed by the application of the lysate supernatant. Non-specific and unbound proteins were removed by washing the column with 20 CV of Buffer B1 and 20 CV of Buffer B2. Proteins were eluted with 11 CV Buffer C in 1 mL fractions. Columns were regenerated by rinsing with 10 CV of Buffer D. 1 mL-samples of the column flow through after sample addition (flow through, FT), Buffer B1 (washout, WI) and Buffer B2 (washout II, WII) were collected, respectively. After three purification cycles, columns were stripped and recharged as follows: First, 20 CV of ddH₂O were added, followed by 5 CV of Stripping Solution, containing the chelating agent ethylenediaminetetraacetic acid (EDTA), to remove the nickel ions. Afterwards, 20 CV of ddH₂O were followed by 3 CV of 100 mM NiSO₄ to recharge the columns. Columns were prepared for storage by applying 20 CV of ddH₂O followed by 20 CV of Storage Solution.

Size-Exclusion Chromatography (SEC) Following Ni-NTA chromatography, the protein solutions were further purified via size exclusion chromatography. This technique allows for separation according to molecular size and was applied in this work for removing oligomers and aggregates of the target proteins as well as low molecular components. The system output is illustrated in a chromatogram, displaying the intensity of absorbance, indicated as absorbance units (AU), over the retention volume or retention time (required volume/time for a protein to elute after injection). The signal intensity is proportional to the concentration of the eluted analyte. In case of optimal separation, multiple peaks or shifts on the chromatogram correspond to different components of the separated sample and can be directly assigned to the molecular size by comparison with a protein standard of known composition. The “peak integration” function of the UNICORN 6.3 control software was used to identify and measure several curve characteristics including peak areas, retention time and peak widths. The required baseline was calculated automatically.

Prior to application, the protein samples were concentrated to a final volume of about 5 mL and were loaded manually via a capillary loop (5 mL) onto a GE Healthcare HiLoad 60/600 Superdex 75 pg column 7 using an AKTA™ Avant chromatography system. Prior to use, the Storage Solution was removed with 1.5 CV ddH₂O. The column was equilibrated and proteins were eluted with 1.5 CV Running Buffer in 2 mL fractions in 96-well DWBs at a flow rate of 1 mL/min. Runs were operated at room temperature (RT) and monitored at a wavelength of 280 nm. Afterwards, the column was re-equilibrated with ddH₂O and Storage Solution. Purity of the proteins was confirmed by SDS-PAGE and the molecular weight (MW) was determined based on the calibration curve provided by GE Healthcare. Desired peak fractions were pooled and concentrated. Aliquots of adequate volumes were snap frozen in liquid nitrogen and stored at −80° C.

Buffers and solutions used for size-exclusion chromatography. All buffers and solutions were sterile-filtered (0.2 μm) and degassed prior to use. The pH was adjusted with HCl.

### Buffer/Solution Composition

Running Buffer 20 mM Tris, 150 mM NaCl, pH 7.5
Storage Solution 20% Ethanol

### Buffer Exchange and Protein Concentration

Prior to SEC, protein samples eluted from Ni-NTA gravity flow columns were buffer exchanged and concentrated, in order to remove remaining imidazole present in the Elution Buffer. Buffer exchange was realised by dialysis. The protein solution was transferred into a Spectra Por® 3 Dialysis Membrane with a molecular weight cut off (MWCO) of 3500 Da (Spectrum Laboratories) and incubated overnight at 4° C. in 5 L Running Buffer under continuous stirring. Dialysed proteins were applied to Vivaspin® 6 Centrifugal concentrator columns, 5000 Da MWCO (Sartorius) and centrifuged to get a final volume of about 5 mL. After performing SEC, the protein pools were concentrated via Vivaspin® 20 Centrifugal concentrator columns, 5000 Da MWCO (Sartorius).

### Generation of Immuno-Affinity Chromatographic Columns

In the scope of this invention, novel immuno-affinity columns for antibody purification were developed. The previously described TtSlyD-Fc-III-4C scaffold-affinity peptide chimers served as a capture molecule/ligand for IgGs and were permanently coupled to a chromatographic column matrix.

For immuno-affinity chromatography, in brief, the crude solution containing the desired antibody (e.g. cell culture supernatant) is applied to the column. The antibody is captured by the affinity ligand comprised in the column matrix; contaminants are removed by intensive washing, and the immunoglobulins are eluted by adding an appropriate buffer.

Immobilisation of the Fc-specific ligands was conducted by covalently coupling primary amino groups to N-Hydroxysuccinimide (NHS)-activated highly cross-linked agarose beads, comprised in HiTrap NHS-activated HP columns (1 mL, GE Healthcare). The coupling procedure was performed according to the manufacturer's protocol. Required buffers are indicated below. The ligands were dissolved in Standard Coupling Buffer and concentrated to 1 mL, leading to a final concentration of 0.5-10 mg/mL. The column was washed with ice-cold 1 mM HCl to remove the isopropanol present in the manufacturer's storage buffer. 1 mL of the ligand solution was injected manually and the column was incubated for 30 min at 25° C. Afterwards the column was connected to the chromatographic system (AKTA™ Avant, GE Healthcare) and the ligand solution was washed out with 3 CV of Standard Coupling Buffer collecting the column flow through. To deactivate any excess active groups that had not coupled to the ligand and to wash out non-specifically bound ligands, 6 CV of Buffer A, 6 CV of Buffer B and another 6 CV of Buffer A were injected. The washout was collected and the column was incubated for 30 min at RT. Thereafter, 6 CV of Buffer B, 6 CV of Buffer A and 6 CV of Buffer B were injected and the washout was collected alike. Finally, the pH was adjusted by applying 10 CV of Binding Buffer. In case the column was not used straight away, it was rinsed with 5 CV of Storage Buffer and stored at 8° C.

Buffers used for the generation of immuno-affinity chromatographic columns. αAll buffers were sterile-filtered (0.2 μm) and degassed prior to use. The pH was adjusted either with HCl or NaOH.

### Buffer Composition

Standard Coupling Buffer 0.2 M NaHCO₃, 0.5 M NaCl, pH 8.3
Buffer A 0.5 M Ethanolamine-HCl, 0.5 M NaCl, pH 8.3
Buffer B 0.1 M NaOAc, 0.5 M NaCl, pH 4
Storage Buffer 0.05 M Na₂HPO₄, 0.1% NaN₃, pH 7
Binding Buffer 20 mM Tris, 150 mM NaCl, pH 7.5. The Binding Buffer corresponds to the SEC Running Buffer. Purified scaffold proteins were dissolved in this buffer for long term storage.

To assess the maximal coupling capacity of the column matrix, increasing ligand concentrations from 1.5-3 mg/mL were applied. The flow through containing excessive ligand was analysed via SDS-PAGE and the coupling efficiency was evaluated as follows: PD-10 desalting columns (GE Healthcare) were equilibrated with 25 mL 0.1 M NaH2PO4, 150 mM NaCl, pH 7 (Equilibration Buffer8), followed by addition of 0.5 mL of washout. Elution was performed in a two-step procedure with Equilibration Buffer by first adding 2 mL to remove salt and other low molecular weight components, followed by 1.5 mL to elute the desired high molecular weight proteins. Absorbance of the eluted fraction was measured using a NanoDrop™ OneC microvolume UV-Vis spectrophotometer (Thermo Fisher Scientific). The coupling efficiency was calculated. See FIG. 2.

### Evaluation of Column Parameters

Chromatographic TtSlyD-Fc-III-4C immuno-affinity columns were generated, and desired parameters such as affinity, chemical stability and reusability were evaluated using a commercial, high pure rabbit Immunoglobulin G (rbIgG) supplied by Sigma-Aldrich. Test runs were performed with an AKTA™ Avant chromatographic system. Prior applying the rbIgG, the affinity column was equilibrated with 20 CV of Equilibration Buffer. The rbIgG was diluted to a final concentration of 1 mg/mL. 1 mL of the sample was injected manually using a 1 mL syringe and the column was incubated for 30 min at 8° C. to allow binding to the ligand. Unbound antibodies were removed by adding 10 CV of Washing Buffer. IgGs were eluted with Elution Buffer over 5 CV in 0.2 mL fractions in a 96-well DWB. To neutralise the eluates, 40 μL of 1 M arginine solution per well were added. Cleaning-in-place (CIP) procedure of the column was performed by first washing with 10 CV of Regeneration Buffer, followed by rinsing with 15 CV 100 mM NaOH. In case the column was not re-used immediately, it was equilibrated in Storage Buffer and stored at 8° C.

Buffers and solutions used for rbIgG purification were as follows. All buffers and solutions were sterile-filtered (0.2 μm) and degassed prior to use. The pH was adjusted either with HCl or NaOH

### Buffer/Solution—Composition

Equilibration Buffer 50 mM Tris-HCl, 0.05% Tween® 20, pH 7.8
Wash Buffer 50 mM Tris-HCl, pH 6.0
Elution Buffer NH₄Ac—AcOH, pH 3.4
Regeneration Buffer NH₄Ac—AcOH, pH 2.2
CIP Solution 100 mM NaOH
Storage Buffer 0.05 M Na₂HPO₄, 0.1% NaN₃, pH 7

### Sodium Dodecyl Sulfate Polyacrylamide Gel Electrophoresis (SDS-PAGE)

Protein fractions were analysed on SDS-PAGE under both, reducing or non-reducing conditions. Samples were mixed with the appropriate amount of NuPAGE® LDS sample buffer (4×) (Thermo Fisher Scientific) and NuPAGE® Reducing agent (10×) (Thermo Fisher Scientific) (for reduced gels) or water (for non-reduced gels) to get a total volume of 50 μL and incubated for 10 min at 95° C. in a thermocycler. Gel runs were performed with 15 μL/10 μg sample per lane on NuPAGE® Bis-Tris 4-12% Gels (Thermo Fisher Scientific), alongside 5 μL Novex™ Sharp pre-stained protein ladder (Thermo Fisher Scientific) to estimate the molecular weight of protein bands. Electrophoresis was performed at a constant voltage of 200 V. Gels were stained with Instant Blue™ Protein stain (Expedeon) and de-stained overnight in ddH₂O. Images were taken in a ChemiDoc MP™ device (Bio-Rad).

### Spectrophotometric Determination of Protein Concentration

The concentration of purified protein after SEC was determined spectrophotometrically using a NanoDrop™ OneC microvolume UV-Vis spectrophotometer (Thermo Fisher Scientific). The appropriate dilution buffer served as Blank. Measurements were performed with a general reference setting based on a 0.1% (1 mg/mL) protein solution producing an absorbance at 280 nm of 1.0 A (where the path length is 1 cm). The protein concentration was calculated according to the Beer-Lambert law, with subsequent consideration of the specific protein absorbance at 280 nm. The corresponding absorbance units (AU) were calculated by the Vector NTI(Thermo Fisher Scientific) software.

Surface plasmon resonance (SPR) The affinities and binding kinetics of the generated TtSlyD-Fc-III-4C mutants were evaluated by SPR using a Biacore biosensensor system. The Biacore system enables a real-time monitoring of intermolecular interactions between the ligand, immobilised on a sensor chip gold-surface, and the analyte which is free in solution and passes over the ligand (Healthcare, G., Biacore™ Assay Handbook 29-0194-00 Edition AA). Interaction of the binding partners leads to SPR signals (responses), measured in resonance units (RU) and illustrated as plot against time (sensorgram).

Regarding the procedure, in brief, the analyte is injected on the chip, interacting with the immobilised ligand. The change in concentration of molecules at the chip surface leads to a change in the refractive index, monitored as response unit (RU). Dissociation of analyte is triggered by continues buffer flow, followed by complete removal (chip regeneration) and starting of a new analysis cycle. The obtained SPR data were fitted to a mathematical model in order to derive the kinetic parameters, such as the association rate constant ka (M-1s-1) and the dissociation rate constant kd (s-1).

Interaction kinetics are examined by monitoring different analyte concentrations over time. Kinetic parameters, such as association (ka) and dissociation rate constants (kd) are evaluated in relation to a mathematical model. One model applied was the ″′ Langmuir interaction model″′ (O'Shannessy, D. J., et al., Determination of rate and equilibrium binding constants for macromolecular interactions using surface plasmon resonance: use of nonlinear least squares analysis methods. Anal Biochem, 1993. 212(2): p. 457-68), assuming a 1:1 interaction where one ligand molecule interacts with one analyte molecule. The kinetic model of “Bivalent analyte binding” assumes a bivalent analyte, where one analyte molecule can bind to one or two ligand molecules. The kinetic analysis experiments, performed within this invention, were carried out as multi-cycle kinetics, testing each analyte concentration in a single cycle and regenerating the chip surface after each cycle. A successful regeneration implies the removal of the bound analyte without detaching the ligand or restricting its activity. Thus, a regeneration scouting was performed prior to the kinetic measurements.

### Regeneration Scouting

Regeneration scouting was carried out using a Biacore 3000 system and a CM5 chip provided by GE Healthcare. The chip exhibits carboxymethylated dextran covalently attached to a gold surface. Ligands were immobilised on the chip surface via amine coupling, trying to achieve ligand densities around 3000 RU. The analyte (human IgG, 300 nM) was diluted in Sample Buffer. Regeneration was tested according to the following protocol. In a first cycle, 300 nM human IgG was injected. Bound antibody was displaced by addition of System Buffer and two 1 min pulses of 10 mM Glycine pH 2. This cycle was repeated six times, followed by two additional cycles using 10 mM Glycine, pH 1.75 and pH 1.5, respectively.

### Operation Program for Regeneration Scouting

| Injection | Flowrate [μL/min] | Volume [μL] |
|---|---|---|
| I. Cycle (6x) | | |
| Human IgG (300 nM) | 30 | 90 |
| System Buffer | 20 | 5 |
| 10 mM Glycine pH 2 | 20 | 20 |
| 10 mM Glycine pH 2 | 20 | 20 |
| II. Cycle (6x) | | |
| Human IgG (300 nM) | 30 | 90 |
| System Buffer | 20 | 5 |
| 10 mM Glycine pH 2 | 20 | 20 |
| 10 mM Glycine pH 2 | 20 | 20 |
| III. Cycle (6x) | | |
| Human IgG (300 nM) | 30 | 90 |
| System Buffer | 20 | 5 |
| 10 mM Glycine pH 2 | 20 | 20 |
| 10 mM Glycine pH 2 | 20 | 20 |
| Sample Buffer | System Buffer + 1 | |
| | mg/mL CMD | |
| System Buffer | HBS-ET+, pH 7.4 | 10 mM HEPES, 150 mM |
| | | NaCl, 3 mM EDTA, 0.5% |
| | | (v/v) Surfactant P20 was |
| | | comprised with 0.005% |
| | | (m/v) Tween ® 20 |

### Kinetic Screening

Kinetic measurements were carried out using a Biacore 3000 and Biacore 8K system with a C1 chip (GE Healthcare). The chip assembly is similar to the C5 chip except for lacking the dextran threads. Scaffolds were immobilised via amine coupling, i.e. proteins were covalently linked to carboxymethyl groups present on the chip surface. The chip surface was flushed with 270 nM analyte prior to measurement in order to saturate remaining free binding sites on the chip surface (conditioning) and regenerated with Glycine buffer, pH 1.75. In brief, the scaffold proteins were immobilised on a Biacore C1 chip via amine coupling. IgG was injected in different concentrations and the chip was regenerated after each cycle.

### Immobilisation of Ligand

Desired ligands were diluted in Sample Buffer to a final concentration of 5 μg/μL. The chip surface was purged with two one-minute injections of Wash Buffer and finally primed with System Buffer. Reactive groups were activated by applying 40 μL NHS/EDC (50% mix) with a flow rate of 20 μL/min. Ligand solutions were injected with a flow rate of 50 μL/min to achieve RU responses suitable for the subsequent performances. (RU values around 100 were not exceeded, in order to avoid steric crowding effects at high analyte concentrations). The excess of NHS-activated esters was quenched by chip exposure to 100 μL of a 1 M ethanolamine hydrochloride (EA-HCl) solution (pH 8.5) at a flow rate of 20 μL/min.

### Kinetic Screening

The analyte was diluted stepwise in Sample Buffer to achieve desired concentrations. IgG (human IgG or rabbit IgG, Sigma-Aldrich) at indicated concentrations was injected over the chip at 60 μL/min or 40 μL/min and 25° C. or 37° C., respectively. At the end of each analytical cycle chip surface was regenerated with two 1 min pulses of 10 mM glycine pH 1.75.

### Results

In the present invention, the peptide-grafting was realised by generating a DNA vector construct, encoding the FKBP domain of the TtSlyD protein together with the Fc-III-4C peptide. The appropriate sequences were inserted into the prokaryotic expression vector pQE80-Kan, allowing for DNA amplification and expression in E. coli. IgG-affinity-columns were generated by coupling the recombinantly-produced TtSlyD-Fc-III-4C to a commercially available NHS-sepharose matrix. Proof-of-principle purification experiments were performed with a highly pure IgG solution. The chemical stability and reusability of the column matrix was further evaluated by exposure to extreme alkaline conditions and multiple repetitions of column runs. Maturation of the Fc-binding part, with respect to the goal of facilitating milder elution conditions, was realised via Quick change PCR (QC-PCR).

Tryptophan 11 which plays a key role in the interaction of the peptide with the Fc-part of IgG was substituted by twelve amino acids exhibiting different biochemical properties. Expression levels and solubility of the different variants were pre-tested in 96-well-format.

Selected mutants were expressed in mid-scale format (250 mL) and the TtSlyD-FcIII-4C scaffolds were purified via immobilised metal-ion affinity chromatography (IMAC) and size-exclusion chromatography (SEC). To simplify expression and purification of the potential column ligands, a chemically synthesised Fc-III-4C peptide library, lacking the bridge forming cysteines, was created and screened for high-affinity variants in a HT-NimbleGen microarrayThe ten best binders were identified, grafted onto the TtSlyD backbone, and expressed and purified. The interaction of the generated protein variants with the IgG was evaluated by kinetic screenings using the surface plasmon resonance (SPR) technology.

Grafting of the Fc-III-4C peptide onto the FKBP domain of the Thermus thermophilus SlyD protein was realised by molecular cloning. Sequence analysis revealed the correct sequence of the final pQE80Kan-TtSlyD-Fc-III-4C expression construct.

The TtSlyD-scaffold, carrying the original unaltered Fc-III-4C insert, was expressed in 250 mL scale followed by purification via immobilised metal-ion affinity (IMAC) and preparative size-exclusion chromatography (SEC). In a proof-of-principle experiment, the protein was coupled to a column matrix in order to assess its potential as affinity ligand for IgG-FPLC purification.

Ligand-matrix combination was performed by covalently coupling primary amine groups, present in the scaffold protein, to NHS-activated highly cross-linked agarose beads, comprised in HiTrap NHS-activated HP columns (1 mL, GE Healthcare). Primary amine groups are found in Lysine residues and in the N-terminus of proteins. Lysine residues are predisposed as conjugation points, since they commonly have an exposed position. The TtSlyD FKBP domain possesses five Lysine residues that can potentially react with the NHS ester groups. A coupling efficiency of 93% was achieved.

Attaching the IgG-Fc-affinity scaffolds to the column, via random amine coupling, results in a heterogeneous matrix composition. The affinity peptide loops are not necessarily opposed to the analyte solution. The binding capacity of the final column might be therefore affected or even impaired. A possible countermeasure would be a thiol-directed immobilisation, by introducing a single cysteine residue in the C-terminal part of the scaffold protein, and subsequent coupling to a thiol-containing solid matrix (Ljungquist, C., et al., Thiol-directed immobilization of recombinant IgG-binding receptors. Eur J Biochem, 1989. 186(3): p. 557-61). Furthermore, the potential effects of ligand density have to be considered.

In a proof-of-principle experiment, one of the generated NHS-sepharose-TtSlyD-Fc-III-4C columns was tested with respect to its IgG-binding capacity. The rabbit IgG purification via NHS-sepharose-TtSlyD-Fc-III-4C columns showed no ligand leakage. The results are in accordance with the findings as described in Gong et al. (Gong, Y., et al., Development of the Double Cyclic Peptide Ligand for Antibody Purification and Protein Detection. Bioconjug Chem, 2016. 27(7): p. 1569-73), who have shown enrichment efficiencies of rabbit IgG on Fc-III-4C -agarose beads comparable to Protein A beads, at the selected pH, which successfully demonstrates the competitive IgG-binding capability of scaffold-affinity chimers, compared to commercially available affinity matrices.

To further assess chemical stability of the ligand and reusability of the column matrix, the purification process was automated and the scope of the protocol was reduced, in order to run several cycles in a row. The yield of eluted antibody was almost the same for all runs, as verified by the spectrophotometric determination of protein concentration and the monitored peak area. Even after 30 cycles, reproducible chromatograms without significant peak shifts or broadening could be recorded.

In order to test for an improvement of IgG elution conditions by engineering the Fc-III-4C binding site, the Fc-binding part was altered by QC-PCR, with the intent to selectively screen for protein variants that exhibit the following features: A fast association rate, favorable in terms of antibody capture and a fast dissociation rate, allowing for antibody detachment under relatively mild pH conditions.

Tryptophan, one of the main driver of binding and strongest interacting molecule between the Fc-III-4C and the IgG-Fc-part, was exchanged against the twelve amino acids glycine, serine, alanine, arginine, lysine, glutamic acid, lysine, threonine, asparagine, glutamine, tyrosine or histidine. The introduction of histidine residues has already been successfully applied for engineering affinity-ligands in terms of lowering the binding strength (Watanabe, H., et al., Optimizing pH response of affinity between protein G and IgG Fc: how electrostatic modulations affect protein-protein interactions. J Biol Chem, 2009. 284(18): p. 12373-83). The dissociation of the antibody under relative mild acidic conditions is hereby promoted by the electrostatic repulsion between the ligand and the antibody. Sequence analyses confirmed the successful insertion of all desired mutations.

For further purification attempts, expression of all protein variants was carried out in 250 mL format as described above. After harvesting the cells, the cell pellets were chemically disrupted, centrifuged, sterile filtered, and the soluble fractions were applied to Ni-NTA gravity flow columns. Desired proteins were eluted by dissolving the binding of the polyhistidine-tag, present in the scaffold proteins, to the nickel column with a fixed concentration of 250 mM imidazole. Monomeric fractions were pooled and used for further analyses. The proportion of obtained protein monomers in relation to the amount of initially loaded protein ranged from 12% (W to A mutation) to 97% (wild type). The results show that the tryptophan at position 11 is crucial for the stability of the monomeric protein. The aim of alternating the Fc-binding site was, to generate a protein variant that is characterised by a high sufficient affinity and shows at the same time a fast association/dissociation, in order to enable a protein elution under mild pH conditions for the subsequent IgG purification process. Nevertheless, all tested variants revealed affinities and association/dissociation rates in the same ranges.

In additional experiments the cysteines were replaced by alternative amino acids, in order to investigate whether these linkages are essential for the loop stability and protein's affinity towards the IgG-Fc.

Cysteine residues of the Fc-III (DCAWHLGELVWCT, Seq ID NO: 17) and Fc-III-4C (CDCAWHLGELVWCTC, SEQ ID NO: 1) peptide were randomised and the obtained cyclic peptide libraries were tested for bindind against a mAb in a high-throughput NimbleGen microarray. For the Fc-III peptide only one high affinity variant could be identified, namely the wild type peptide.

For the Fc-III-4C, 299 variants that are excelling the IgG-Fc affinity of the wild type peptide were identified. However, among all these high-affinity variants, only sequences where both internal cysteines remained intact were found. Variants, lacking the internal cysteines, all belonged to the low-intensity variants, indicating that the internal cysteines are absolutely mandatory. The terminal disulfide bridge, in contrast, is not necessary for preserving the peptide's affinity towards IgG, the binding strengths could even be improved in some cases.

Several positions could be identified, in which an aa-exchange led to altered intensities and binding affinities. The most notable position was the leucine residue at position 7 in Fc-III-4C. The respective replacement by glutamine significantly improved the affinity. Ten of the high-affinity variants were grafted onto the TtSlyD FKBP domain and further investigated in terms of purification profiles and IgG-Fc affinities. The variants, lacking the external cysteines, are called Fc-III-XC. Analysed were only the first 50 variants exhibiting the highest affinities against the anti-CD44 antibody. At the 3′ end a clear tendency towards the acidic amino acids aspartic acid and glutamic acid as well as the polar amino acid asparagine is recognisable. However, the most abundant cysteine substitution at the C-terminus of the peptide is proline. Prolines have a low conformational freedom and might therefore stabilise the loop structure, resulting in a higher affinity. 5′-substitutions occur rather randomly, there is no clear trend bservable. In most of the cases the cysteine is simply deleted, or exchanged by the same amino acids as named above. The sequencing data revealed correct sequences for all desired constructs.

As expected, the TtSlyD-Fc-III-XC mutants are less prone to aggregation compared to the variants exhibiting four cysteine residues. Molecular interactions and affinities of the cysteine deficient protein variants, were tested for both, rabbit IgG and human IgG, at different temperatures (25° C. and 37° C.).

KD values in the range of 8-43 nM were obtained in the interaction analyses with hIgG. For rbIgG, the KD values ranged between 15 and 52 nM. According to Gong et.al. the KD values of the chemically synthesised Fc-III-4C peptide were 2.45 nM for human and 5.67 nM for rabbit IgG, representing a higher affinity towards Fc than the natural Protein A binder. TtSlyD-Fc-III-2C Hit No. 4 was identified as the most promising candidate for IgG purification via FPLC. It exhibits a fast association rate (t/2 diss=36 min.) and a relatively high binding affinity (K_{A}=1.09×10⁸ 1/M) (measurements for hIgG at 25° C.). The covalent attachment of the affinity protein to a matrix, e.g. a sepharose resin, will reveal its full potential as chromatographic ligand.

1. A chimeric IgG-Fc-binding ligand polypeptide, comprising a protein fragment of SlyD, wherein the IF domain thereof is replaced by the affinity peptide Fc-III-4C (CDCAWHLGELVWCTC, SEQ ID NO: 1) or a Fc-III-XC variant thereof (X₁DCAWHLGELVWCTX₂, SEQ ID NO: 3), wherein X₁ is either missing or independently selected from the group of C, D, P, E, and K, and X₂ is independently selected from the group of C, Q, P, and E.

2. The chimeric IgG-Fc-binding ligand polypeptide according to claim 1, wherein said SlyD is from a Thermus species.

3. The chimeric IgG-Fc-binding ligand polypeptide according to claim 1, comprising the sequence
(SEQ ID NO: 5)
MKVGQDKVVTIRYTLQVEGEVLDQGELSYLHGHRLIPGLEEALEGREEG
EAFQAHVPAEKAYCDCAWHLGELVWCTCGKDLDFQVEVVKVREATPEEL
LHGHA.

4. The chimeric IgG-Fc-binding ligand polypeptide according to claim 1, further comprising a C-terminal amino acid tag.

5. The chimeric IgG-Fc-binding ligand polypeptide according to claim 1, wherein said polypeptide exhibits binding with high affinity to IgG species selected from the group consisting of human, rabbit, mouse, rat, pig, goat, horse, and bovine.

6. A divalent binder molecule, comprising two fused chimeric IgG-Fc-binding ligand polypeptides according to claim 1.

7. The chimeric IgG-Fc-binding ligand polypeptide according to claim 1, coupled to a solid carrier.

8. The coupled ligand according to claim 7, wherein said coupling is through the lysine side chains of the SlyD with NHS esters comprised in a sepharose matrix.

9. A method for producing the chimeric IgG-Fc-binding ligand polypeptide according claim 1, comprising recombinant expression of said ligand polypeptide in a suitable host cell or comprising a chemical synthesis of said ligand polypeptide.

10. The method according to claim 9, further comprising the step of coupling the chimeric IgG-Fc-binding ligand polypeptide to a solid carrier.

11. A method for purifying an immunoglobulin, comprising contacting a solid carrier having the chimeric IgG-Fc-binding ligand polypeptide according to claim 1, and suitably eluting said immunoglobulin from said chimeric IgG-Fc-binding ligand polypeptide.

12. The method according to claim 11, comprising fast protein liquid chromatography (FPLC).

13. The method according to claim 11, wherein said elution conditions for said immunoglobulin are milder compared to a solid carrier material comprising protein A coupled thereto.

14. The method according to claim 11, comprising a chemical regeneration step for said solid carrier material using harsher conditions compared to a solid carrier material comprising protein A coupled thereto.

15. (canceled)

16. The chimeric IgG-Fc-binding ligand polypeptide according to claim 2, wherein the Thermus species is Thermus thermophiles.

17. The chimeric IgG-Fc-binding ligand polypeptide according to claim 4, wherein the C-terminal amino acid tag is a His₈ tag.

18. The divalent binder molecule according to claim 6, wherein the two fused chimeric IgG-Fc-binding ligand polypeptides are fused head-to-tail with each other.

19. The chimeric IgG-Fc-binding ligand polypeptide according to claim 7, wherein the solid carrier is a bead and/or column matrix.

20. The method according to claim 9, wherein the host cell is E. coli.

21. The method according to claim 10, wherein the solid carrier is a bead and/or column matrix.


SEQUENCE LISTING (17 sequences)

SEQ ID NO: 1 - protein, 15 aa, synthetic construct
  REGION 1..15: note = affinity peptide
CDCAWHLGELVWCTC

SEQ ID NO: 2 - protein, 89 aa, Thermus thermophilus
  VARIANT 63: note = Xaa is an amino acid to be displayed
MKVGQDKVVTIRYTLQVEGEVLDQGELSYLHGHRLIPGLEEALEGREEGEAFQAHVPAEKAYXGKDLDFQVEVVKVREATPEELLHGHA

SEQ ID NO: 3 - protein, 15 aa, synthetic construct
  REGION 1..15: note = affinity peptide
  VARIANT 1: note = Xaa can be missing or is C, D, P, E, or K
  VARIANT 15: note = Xaa is C, Q, P, or E
XDCAWHLGELVWCTX

SEQ ID NO: 4 - protein, 13 aa, synthetic construct
  REGION 1..13: note = affinity peptide
DCAWHLGELVWCT

SEQ ID NO: 5 - protein, 103 aa, synthetic construct
  REGION 1..103: note = chimeric ligand peptide
MKVGQDKVVTIRYTLQVEGEVLDQGELSYLHGHRLIPGLEEALEGREEGEAFQAHVPAEKAYCDCAWHLGELVWCTCGKDLDFQVEVVKVREATPEELLHGHA

SEQ ID NO: 6 - protein, 14 aa, synthetic construct
  REGION 1..14: note = affinity peptide
  VARIANT 14: note = Xaa is C, Q, P, or E
DCAWHLGELVWCTX

SEQ ID NO: 7 - protein, 15 aa, synthetic construct
  REGION 1..15: note = affinity peptide
  VARIANT 15: note = Xaa is C, Q, P, or E
CDCAWHLGELVWCTX

SEQ ID NO: 8 - protein, 15 aa, synthetic construct
  REGION 1..15: note = affinity peptide
  VARIANT 15: note = Xaa is C, Q, P, or E
DDCAWHLGELVWCTX

SEQ ID NO: 9 - protein, 15 aa, synthetic construct
  REGION 1..15: note = affinity peptide
  VARIANT 15: note = Xaa is C, Q, P, or E
PDCAWHLGELVWCTX

SEQ ID NO: 10 - protein, 15 aa, synthetic construct
  REGION 1..15: note = affinity peptide
  VARIANT 15: note = Xaa is C, Q, P, or E
EDCAWHLGELVWCTX

SEQ ID NO: 11 - protein, 15 aa, synthetic construct
  REGION 1..15: note = affinity peptide
  VARIANT 15: note = Xaa is C, Q, P, or E
KDCAWHLGELVWCTX

SEQ ID NO: 12 - protein, 15 aa, synthetic construct
  REGION 1..15: note = affinity peptide
  VARIANT 1: note = Xaa is C, D, P, or E
XDCAWHLGELVWCTC

SEQ ID NO: 13 - protein, 15 aa, synthetic construct
  REGION 1..15: note = affinity peptide
  VARIANT 1: note = Xaa is C, D, P, or E
XDCAWHLGELVWCTQ

SEQ ID NO: 14 - protein, 15 aa, synthetic construct
  REGION 1..15: note = affinity peptide
  VARIANT 1: note = Xaa is C, D, P, or E
XDCAWHLGELVWCTP

SEQ ID NO: 15 - protein, 15 aa, synthetic construct
  REGION 1..15: note = affinity peptide
  VARIANT 1: note = Xaa is C, D, P, or E
XDCAWHLGELVWCTE

SEQ ID NO: 16 - protein, 15 aa, synthetic construct
  REGION 1..15: note = affinity peptide
EDCAWHLGELVWCTE

SEQ ID NO: 17 - protein, 13 aa, synthetic construct
  REGION 1..13: note = affinity peptide
DCAWHLGELVWCT